(19) United States
(12) Patent Application Publication
(10) Pub. No.: US 2010/0179537 A1
(43) Pub. Date: Jul. 15, 2010

(54) ABLATION FOR ATRIAL FIBRILLATION

(76) Inventor: Rassoll Rashidi, Lakewood, OH (US)

Correspondence Address: FAY SHARPE LLP, 1228 Euclid Avenue, 5th Floor, The Halle Building, Cleveland, OH, 44115, US

(21) Appl. No.: 12/729,917

(22) Filed: Mar. 23, 2010

Related U.S. Application Data
(63) Continuation of application No. 11/525,476, filed on Sep. 22, 2006.

(51) Int. Cl. A61B 18/14

(52) U.S. Cl. 606/34

(57) ABSTRACT

A probe operates in conjunction with an ablation system to prevent accidental injury of the esophagus during atrial ablation procedures. A distal portion of the probe is placed into the esophagus via the nasal cavity and positioned in the region of the esophagus that is in contact with the left atrium. Regulated cooling fluid with desired temperature and pressure continuously circulates from the external source of the related device into a sac of the probe. Temperature and pressure sensors are disposed within the sac of the probe to transmit data to the external related devices of this invention. The information from the sensors within the sac of the probe can provide a safety feature to control or stop the energy delivery from the ablation energy generator (i.e., radio frequency generator) and to prevent the advancement of the lesion formation that is created by the tip of the ablation catheter in the left atrium.

This application is a continuation application of U.S. Ser. No. 11/525,476, filed Sep. 22, 2006, now abandoned.

### BACKGROUND OF THE INVENTION

The present invention relates to a probe with its accessory devices employed to operate in conjunction with an ablation system to prevent the accidental injury of the esophagus during atrial ablation procedures, for example, to control the propagation and advancement of lesion formation.

A primary device for monitoring the live body intra-cavity tissue temperature and cooling and/or controlling the intra-cavity tissue is presented in U.S. Pat. Nos. 4,601,296 and 4,497,324, 4,375,220-4,010,795. A distal portion of the device includes a probe and/or catheter that can be inserted to an intra-body cavity or transvenously placed to a desired position of the heart or other organ. A distal portion of the probe/catheter commonly includes a temperature sensor (i.e. thermistors, thermocouples) and/or heat transfer member.

Typically, known cooling systems that are utilized to cool the intra-body cavity or tissue comprise a refrigerator, a pump, and a probe (U.S. Pat. No. 4,249,923 issued to Walda). The probes are commonly elongated, flexible, cylindrical bilumen tubes having a distal portion and a proximal end. A heat exchanger member is disposed and connected to the bilumens of the probe at the distal portion. One of the lumens is the inlet and other lumen is the outlet for fluid circulation from the fluid refrigerator to the heat exchanger member at the distal portion of the probe.

The proximal ends of the bilumen tube are connected to the fluid refrigerator and pump. The pump circulates the coolant fluid from the refrigerator to the heat exchanger member of the probe via the inlet and outlet of the probe lumens.

Temperature monitoring catheters typically comprise an elongated, flexible, cylindrical, and electrically non-conductive shaft having a distal end and a proximal end. Heat sensors (i.e. thermistors, thermocouples) are disposed in the wall of the catheter (see, for example, U.S. Pat. No. 4,497,324 issued to Sullivan).

The distal portion of the catheter—where the sensors are—is normally disposed and positioned to the body intra-cavity or transvenously to a desired location where the temperature needs to be monitored. Electrical wires are disposed within the catheter lumen longitudinally and extend to the proximal end. The electrical wires are connected to the sensor(s) in one end and extend to the proximal end of the catheter. The temperature of the body intra cavity can be measured and monitored via the electrical wires by a monitoring/recording device.

A need exists for an improved probe, and probe with accessory devices, to operate in conjunction with an associated ablation system to prevent accidental injury to the esophagus during atrial ablation procedures.

### BRIEF SUMMARY OF THE INVENTION

A device operates in conjunction with an ablation generator and system during the cardiac ablation procedures. The device controls the advancement of lesion formation and propagation. More specifically, the device protects a desired depth of viable myocardium tissue directly in front of lesion formation on the epicardial side.

Further, a probe operated by the device includes a sac at a distal portion. Fluid with desired temperature and pressure/volume will be circulated continuously in the sac from the device. The distal portion of the probe is positioned via the nasal cavity into the esophagus right behind the left atrium. The cooling fluid in the sac can protect the esophagus from the injury during the left atrium ablation procedure.

An accessory device can include an expandable heat exchanger sac (compliant or non-compliant) disposed at the distal portion of the probe. Fluid is circulated continuously in the sac from an external source (device) with the desired temperature and pressure/volume.

An external source of hot, cold, and reservoir fluid tanks provide continuous fluid circulation into the probe heat exchanger or sac.

A series of pumps connected in line with the external tanks (fluid sources) in the device and the probe provide desired pressure in the probe heat exchanger or sac for the non-compliant sac and desired volume for the compliant elastic sac with continuous fluid circulation.

A compliant heat exchanger sac is provided at the distal portion of the probe. Fluid is circulated continuously in the sac with desired volume and temperature from external tanks or fluid sources of the device.

A series of hydroelectrical valves are connected with external tanks or fluid sources, pumps, and probe heat exchanger sac.

A feedback control system such as a microprocessor receives information from the sensors of the device (e.g., pressure, temperature, volume) and probe, and sends commands to the hydroelectrical valves and the fluid circulating pumps and flow meters to ensure the desired pressure and temperature for the fluid in the probe heat exchanger sac.

A temperature control unit is provided for the sac.

The selected desired temperature of the fluid in the sac is correlated quantitatively with data collected from a series of experiments with the depth of the viable tissue adjacent to the probe heat exchanger or sac during ablation procedure (in vitro and/or in vivo).

Another embodiment uses an air probe positioned into the esophagus via a patient's nasal cavity and transfers cooling air or gas from external source to cool the desired segment of the esophagus and return the air/gas back from the esophagus to the external source to allow continuous flow of cooling air/gas in the specific region of the esophagus during atrial ablation procedure. The air probe includes at least three spaced sacs, platinum ring electrodes for use as radio-opaque markers, and temperature sensors at its distal portion. The sacs are typically doughnut-shaped and are disposed like a ring on the distal portion of the probe with some spacing between them.

Another embodiment uses a flexible magnetic probe that is positioned into the esophagus via the nasal cavity. The flexible magnetic probe is preferably constructed with either a permanent magnet or by a flow of electrical current through a magnetic coil within the body of the probe.

An external magnetic field generator provides a variable and/or sufficient magnetic field that can be placed on the side of the chest and is capable of pushing and deflecting the flexible magnetic probe resulting in temporary deflection and dislocation of the esophagus from the heart.

The probe operates in conjunction with an ablation system to prevent accidental injury of the esophagus during atrial ablation procedures. A distal portion of the probe is placed into the esophagus via the nasal cavity and positioned in the region of the esophagus that is in contact with the left atrium.

In one embodiment of this invention the probe comprises an elongated flexible tube with an expandable sac, either compliant or non-compliant, disposed at its distal portion. Regulated cooling fluid with desired temperature and pressure is continuously circulating from the external source of the related device into the sac of the probe. The sac is positioned into the esophagus region that is in contact with the left atrium. Temperature and pressure sensors are disposed within the sac of the probe to transmit data to the external related devices of this invention. The information from the sensors within the sac of the probe can provide a safety feature to control or stop the energy delivery from the ablation energy generator (i.e., radio frequency generator) and to prevent the advancement of the lesion formation that is created by the tip of the ablation catheter in the left atrium. Hence, this can prevent the accidental injury of the esophagus during the left atrium ablation procedure.

In a further embodiment, a distal portion of the probe includes a plurality of in-flow and out-flow perforations within tubes housed in the probe and extends to the proximal end of the probe that is connected to the related external device of this invention. Cooling fluid (liquid, air or other gas) with desired temperature and pressure is delivered from the external device to the out-flow perforations of the distal portion of the probe. The released fluid cools the desired region of the esophagus and will be returned through the in-flow pores of the probe to the external device.

In yet another embodiment of this invention a flexible tubular magnetic probe with a distal end and proximal end is dimensioned for receipt into the esophagus via the nasal cavity. The distal end of the magnetic probe located into the esophagus is temporarily laterally displaced, e.g., laterally pulled or pushed, by an external magnetic field source placed over the side chest of the patient. The tubular flexible magnetic probe is either constructed from a permanent magnet or by applying electrical current in a magnetic coil provided within the probe. The external variable magnetic field source is positioned over the side chest of the patient with convergent angle to have better control over the lateral pushing/pulling of the distal portion of the flexible magnetic probe in the esophagus resulting in temporary displacement and dislocation of the desired region of the esophagus laterally that is in contact with the left atrium. It is important to achieve the temporary lateral dislocation of the esophagus during the left atrial ablation procedure. This prevents the accidental advancement of the lesion formation to the esophagus by the tip of the ablation catheter in the left atrium during the left atrial ablation procedure.

### BRIEF DESCRIPTION OF THE DRAWINGS

FIG. 1 shows a perspective view of the probe presented in this invention.

FIG. 2a and FIG. 2b shows overall view of the components of the system (device) presented in this invention.

FIG. 3 is the schematic view of the probe in the esophagus right behind the left atrium. The probe is connected to the device presented in this invention and ablation generator.

FIG. 4a shows a schematic view of a membrane.

FIG. 4b shows a schematic view of a membrane and associated heat source on one side of the membrane.

FIG. 4c shows a schematic view of a membrane and heat source on one side and a cooling source on the other side of the membrane.

FIG. 5a shows the schematic top view of a sac of the probe positioned in the lower segment of esophagus adjacent to the left atrial wall.

FIG. 5b shows the schematic side view of sac of the probe positioned in the lower segment of esophagus adjacent to the left atrial wall.

FIG. 6 shows a perspective view of the second embodiment of the probe.

FIG. 7 shows an exploded schematic view of the second embodiment of the probe.

FIG. 8 shows the perspective view of yet another embodiment of a flexible magnet probe.

FIG. 9 shows the perspective view of a flexible magnet probe that is exposed in one end to a magnetic field of the same polarity that deflects the probe.

FIG. 10 shows the schematic view of the esophagus and heart.

FIG. 11 shows the schematic view of the esophagus and heart while the flexible magnet is positioned into the esophagus and being displaced or deflected (pushed) by exposure to an external magnetic field of the same polarity resulting in temporary deflection and dislocation of the esophagus from the heart.

### DETAILED DESCRIPTION OF THE INVENTION

Proper propagation of electricity in the right pathway in the heart muscle results in the correct heart muscle contraction and pumping action. When the pathway of electrical propagation in the heart muscle is disturbed by any means, the heart will not contract properly and a patient suffers from heart disease. Abnormality in electrical activities of the atriums (left, right) may result in atrial malfunctions. Atrial malfunction can cause a blood clot in the atrium resulting in a brain stroke or embolism in the lungs and/or an abnormal or irregular heart beat. Abnormality in the atrial electrical activities can also cause chaotic contraction of the atrium muscle known as atrial fibrillation and the other abnormal contraction of the atrium muscle with specific rhythm called atrial flutter.

There are some medical treatments for atrial diseases. One way of treatment is the use of medications. Another way of treating the disease is by performing an ablation procedure on the heart. During the ablation procedure a distal portion of a catheter is transvenously placed into the heart and operators navigate a tip of the catheter in the heart remotely and manually via an actuator on a proximal end of the catheter. The catheter is typically an elongated, non-electrically conductive shaft (with the diameter about 2.5 mm and length approx. 110 cm) with a plurality of spaced ring electrodes (about 1 mm spacing) on the distal portion of the catheter. A platinum dome-shaped electrode (with the diameter about 2.5 mm and length of 4-10 mm) is used as a distal electrode. The catheter electrodes are individually and separately connected to electrical wires within the catheter shaft and that extend to the proximal end of the catheter. The ring electrodes are used to acquire the heart electrical activities that will be conducted to a monitoring and recording device(s). The distal tip electrode of the catheter is utilized to deliver electrical energy from the ablation generator to the tissue adjacent to the electrode.

In the recent years a radio frequency (RF) generator is used for this application. Typically the RF generator utilized for ablation procedures provides a variable energy up to about 50 Watts with the frequency of about 500 kHz. A conductive patch about 10×20 cm is attached to a patient's body. The RF generator is connected to the distal electrode of ablation catheter and patch. RF energy is delivered from the generator to the distal electrode adjacent to the abnormal site of the myocardium (heart muscle) that causes abnormality in the electrical activities of the heart and returns to the patch. When this energy is delivered to the heart muscle it creates a lesion where the distal electrode is in contact with the tissue of the heart. Creation of the lesion is called ablation or destruction of the abnormal tissue of the heart. The energy delivery time is in the order of seconds or minutes (for example, 2 minutes). The longer the duration of energy delivery with a correct amount of energy, the deeper the lesion will be. Typically the depth of the lesion is in the order of few millimeters. The thickness of the heart muscle (myocardium) is also in the order of few millimeters. The lesion depth from the endocardium, where the tip of the catheter is positioned, must be less than that of the thickness of the myocardium. The operator must always leave enough thickness of viable tissue behind the lesion. The destruction of total depth of the myocardium by creation of a lesion during ablation procedure may have deleterious effect on patient.

In the recent years there are more efforts to treat the heart patients that are suffering from atrial fibrillation by the RF ablation procedure rather than treating them by medications. Performance of atrial ablation requires placement of the tip of the catheter into the left atrium transeptally. Some part of a posterior wall of the left atrium is directly in contact with a segment of the esophagus. In order to treat the atrial fibrillation (AF), the operator (cardiologist) typically creates multiple lesions in the wall of the left atrium. These lesions can be in the segment of the atrium that is directly in contact with the esophagus. If the depth of the lesion accidently goes beyond the thickness of the atrial wall by prolonged energy delivery and higher energy, it could damage the esophagus wall. Injury of the esophagus wall can generate an atrial-esophageal fistula which can result in systemic infection and/or death. The operators (cardiologists) that perform atrial ablation are very cautious and careful about that particular region of the atrium that is directly in contact with the esophagus during ablation energy delivery. Although the operators are very careful, however accident and injury of the esophagus can happen. The total thickness of atrial wall and esophagus wall significantly varies from person to person. The range of variance is about 4 mm to 13 mm.

So far, there is no device or method that can provide a quantitative measure to prevent the accidental injury of the esophagus during atrial ablation procedures. This is a procedure that requires great skill.

One embodiment of a system of the present invention includes a probe that will be positioned via the nasal cavity in the esophagus right behind the left atrial wall. The distal portion of the probe comprises an expandable heat exchanger (sac) that can automatically provide accurate temperature to nullify the excessive heat that is transferred into the esophagus from the tip of the catheter.

Another aspect of the system is to temporarily laterally displace and dislocate the esophagus region that is normally in contact with the left posterior atrial wall.

A method of measuring and controlling the temperature of the tissue in front of the lesion formation and lesion propagation opposite to the tip of the ablation probe allows the operator to protect the desired depth of viable tissue of the heart muscle (myocardium).

This method of tissue protection is applied in particular during left atrial ablation procedures.

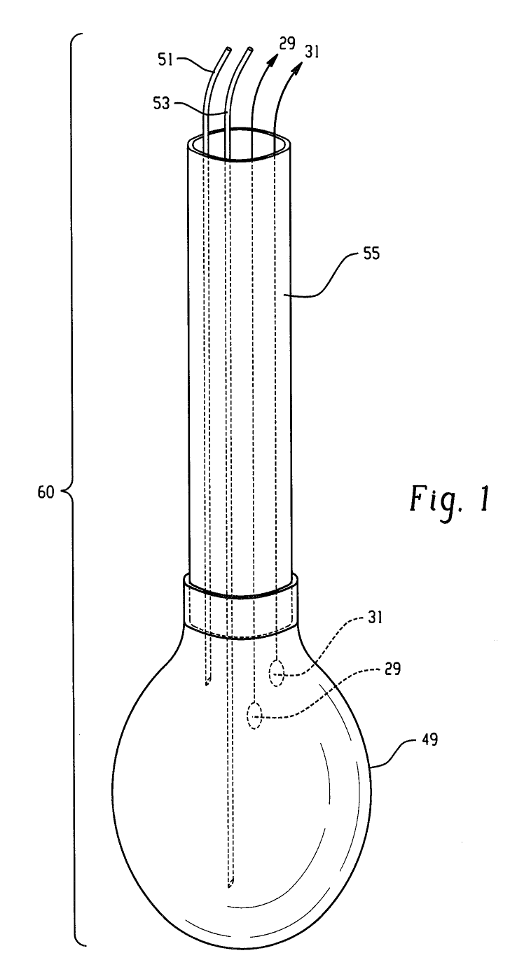

As noted briefly above, a left atrial ablation is performed for the treatment of abnormality in the atrial electrical activities. Referring now to the drawings, which are not intended to limit the invention. FIG. 1 illustrates a perspective view of the embodiment of the probe 60 assembly. The probe of this invention includes an elongated flexible main body 55. The probe can include either a compliant or a non-compliant sac 49 at the distal end. A compliant elastic sac at the distal portion of the probe is utilized for assuming the configuration of the intra-body cavity during a specific procedure. A non-compliant flexible sac is utilized for applying radially controlled pressure into the intra-body cavity during a specific procedure. At least, two flexible lumen tubes 51 and 53 are disposed within the main flexible body 55. The distal ends of tubes 51 and 53 are extended into the sac 49. The proximal ends of tubes 51 and 53 are free. At least one temperature sensor (i.e. thermistor, thermocouple) (29) and one pressure sensor (31) are disposed in sac 49.

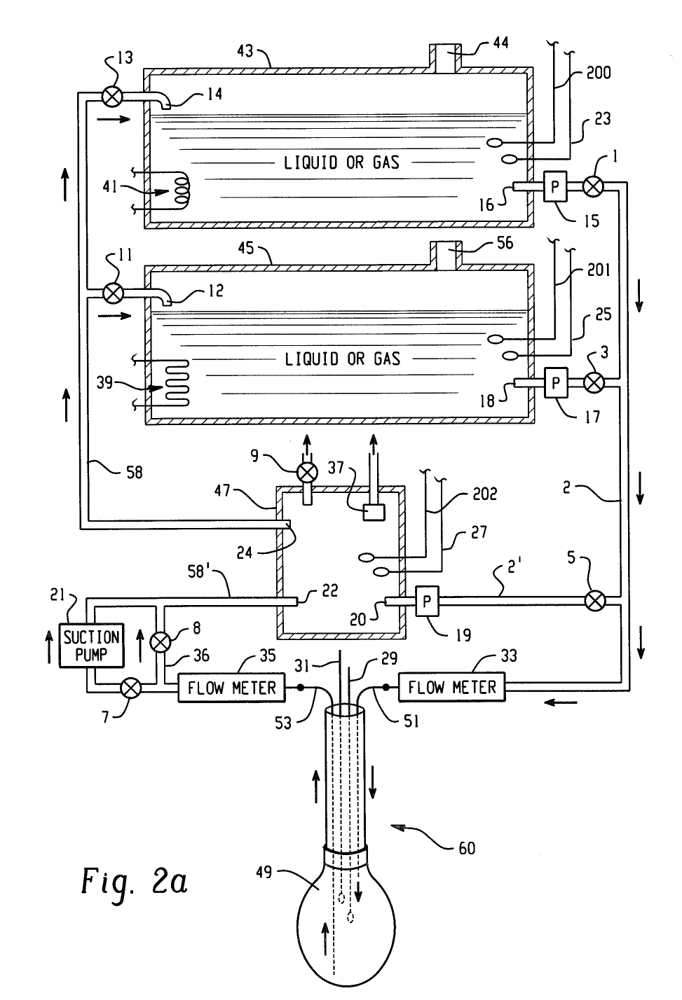

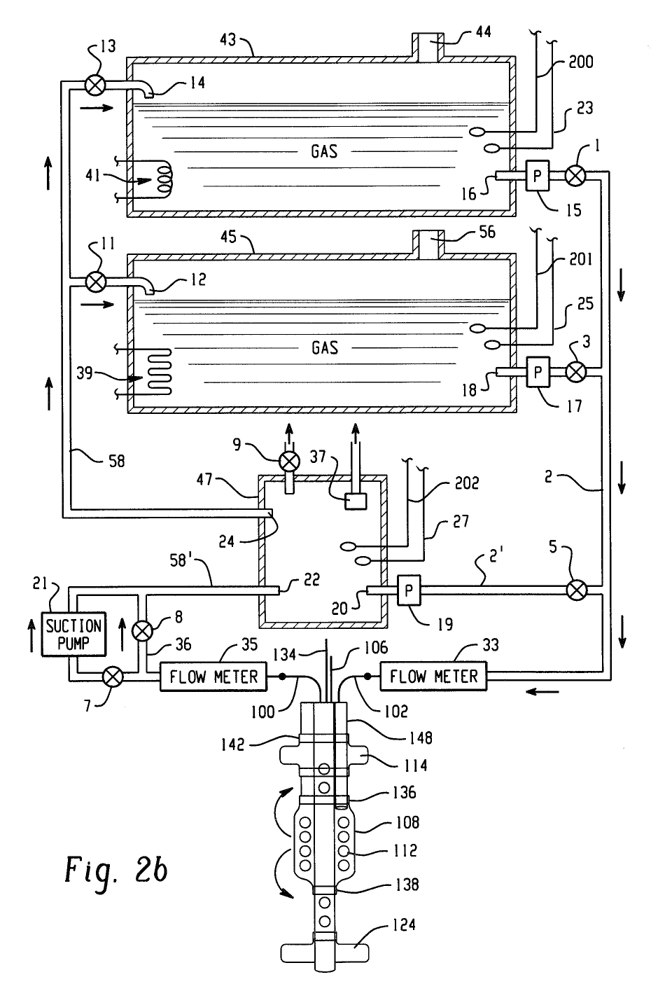

Referring to FIG. 2a, the system includes a hot tank 43 with an opening 44, a temperature sensor 23, pressure sensor 200, heating element 41, liquid inlet 14, liquid (gas) outlet 16, and pump 15. A cooled tank 45 has an opening 56, a temperature sensor 25, pressure sensor 201, cooling coil 39, liquid (gas) inlet 12, liquid (gas) outlet 18, and pump 17. A reservoir 47 comprises inlet 22, outlets 24 and 20, volume meter 37, temperature sensor 27, pressure sensor 202, air vent valve 9, and pump 19. A flow meter 33, flow meter 35, suction pump 21 and disposable probe 60 are also provided. A microprocessor unit 62 in FIG. 3 includes, for example, an ON-OFF switch, START button, temperature selector, temperature indicator, pressure selector, pressure indicator, sac volume selector, and volume indicator. The microprocessor unit is electrically connected to all hydroelectric or gas valves, temperature sensors, heating elements, cooling system, flow meters, volume meter, air vent valve, pressure sensors, pumps, and ablation energy generator.

Again referring to FIG. 2a of this invention, the hot tank, cold tank and reservoir tank are connected to probe 60 by a series of hydroelectric or gas valves 1, 3, 5, 7, 8, 9, 11, and 13 via tubes 2, 2′, 58′ and 58. The device and the system presented in this invention operate or function as follows: first, the device is turned on; and after a few minutes the system is ready for operation. Desired temperature, pressure, volume of the liquid in sac 49 and type of sac (non-compliant or compliant) are all pre-selected. One option is to use a radio-opaque fluid that allows the sac to be viewed by fluoroscopy, although the invention should not be limited to radio-opaque fluids only. A disposable probe 60 is positioned into the esophagus of patient via the nasal cavity. The free ends of tubes 51, 53, temperature sensor 29 and pressure sensor 31 of the disposable probe 60 are connected to the device.

For non-compliant probe sac 49, the switch S of the microprocessor unit (FIG. 3) is turned on. Valves 3, 8, and 11 are opened; pump 17 is turned on, and the liquid is pumped by pump 17 into sac 49 and fills sac 49 and reservoir 47, then returns to tank 45 again. The liquid circulation continues until the temperature of the liquid in sac 49 reaches a pre-selected temperature on the microprocessor unit at which time valves 3 and 11 will be closed and valve 5 will be opened. Pump 19 is turned on and liquid circulation will continue from reservoir tank 47 to sac 49. The function of the reservoir for this device is to keep continuous liquid circulation in sac 49 because the temperature of sac 49 should be the same as the temperature of reservoir 47.

For compliant probe sac 49, the switch S of the microprocessor unit (FIG. 3) is turned on. Valves 3, 7, and 11 are opened; pump 17 and suction pump 21 are turned on. The microprocessor unit with the information from flow meters 33, 35 and volume meter 37 of reservoir determines the amount of preselected liquid volume in sac 49. The temperature of the circulatory liquid of sac 49 is adjusted by hot liquid in tank 43 and cold liquid of tank 45. The liquid volume in the compliant sac 49 is maintained by flow meters 33, 35, tank pumps 15, 17, 19 and suction pump 21 through the microprocessor.

It will be appreciated that in the embodiments of both of FIGS. 2a and 2b, passages 44, 56 may include a valve so that circulating flow to the probe sac 49 is defined as a closed loop. This is to be contrasted with the systems shown in FIGS. 2a and 2b which are presently open. The closed loop arrangement allows the loop to use a sterile fluid if desired.

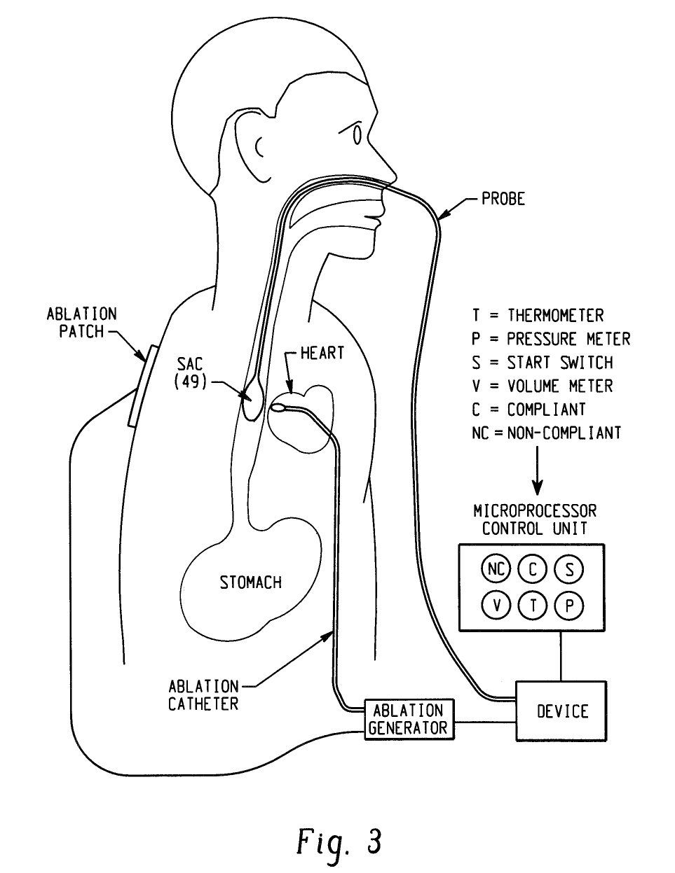

FIG. 4a shows a membrane 70 with the thickness of 80 at temperature T of its surrounding. FIG. 4b illustrates a heat source 72 that approaches membrane 70 from the right side and elevates the temperature of the right side of the membrane 70 from T to T₁, where T₁>T. In the very beginning, the temperature of membrane 70 would be T on the left side and T₁ on the right side. Assuming the heat source 72 remains at the same distance from membrane 70 and continues to heat the membrane 70 with the same amount of energy, the temperature of the left side of the membrane after some time will approach from T to T₁.

Referring to FIG. 4c, further elevation of the temperature at the left side of membrane 70 is precluded by introducing a cooling element source 74. The cooling source 74 from the left controls the temperature gradient in the thickness of membrane 70 within a reasonable range.

FIG. 3 shows a schematic view of the probe presented in this invention. The probe is placed into the esophagus via the nasal cavity. The distal portion of the probe, sac 49 is positioned in the lower esophagus region that is behind the left atrial wall. Cold liquid of adjustable temperature is circulating from the external source (device) into sac 49.

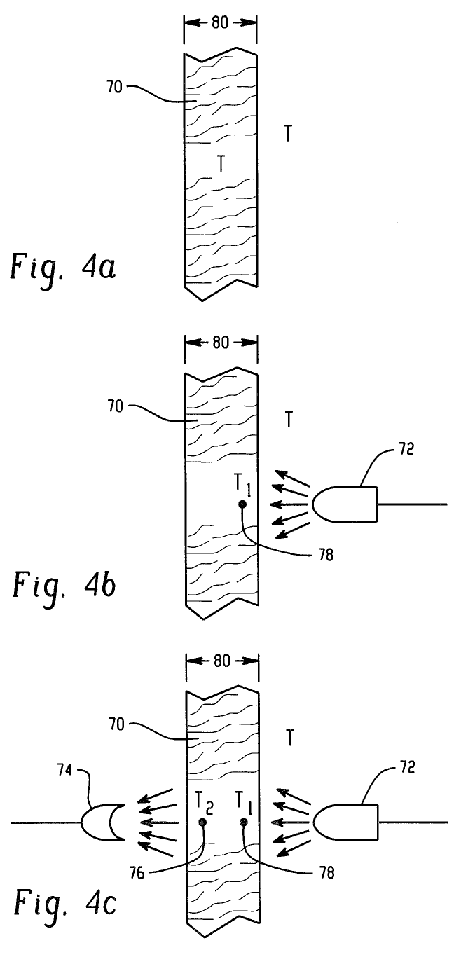

FIGS. 5a and 5b illustrate schematic top and side views of a segment of esophagus wall and left atrial wall. From the right side, the tip of the ablation catheter is positioned on the atrial wall and creates a lesion during ablation energy delivery as shown in FIG. 5a. Sac 49 of the probe is placed in the esophagus directly in front of the ablation catheter tip 85. One skilled in the art will understand that the catheter could be located anywhere in the cross-section of the esophagus since the esophagus has an irregular shape along its length.

The liquid in sac 49 is heated from the tip of the ablation catheter and the temperature of the liquid in the sac 49 will elevate if continuous cooled liquid is not circulated into the sac. The liquid in sac 49 is continuously circulated from the external source. The continuous circulation of liquid with the desired temperature and pressure in sac 49 of the probe is an important feature of this invention. This feature provides even temperature distribution in sac 49 of the probe and results in a more accurate temperature measurement of the esophagus by the temperature sensor of sac 49 during atrial ablation procedure utilizing the system presented in this invention. During the atrial ablation procedure, the cardiologist using the present invention can protect against the accidental injury of the esophagus. For example, this system can automatically shut the ablation generator off in the event of excessive energy delivery by the ablation generator to protect the esophagus from the injury.

During the ablation procedure typically the Radio Frequency (RF) energy (ranging from about 0 to 50 Watts) (FIGS. 5a, 5b) is applied to the distal electrode 85 at the tip of the ablation catheter. Lesion 91 illustrated in FIG. 5a will be formed in the tissue on the front of the catheter tip 85. The depth of the lesion is proportional to the amount of RF energy and duration of the RF energy delivery. As described previously in connection with FIG. 4, the advancement of lesion depth can be stopped by cooling the opposite side of the tissue in front of lesion 91 (see FIGS. 5 a and 5b).

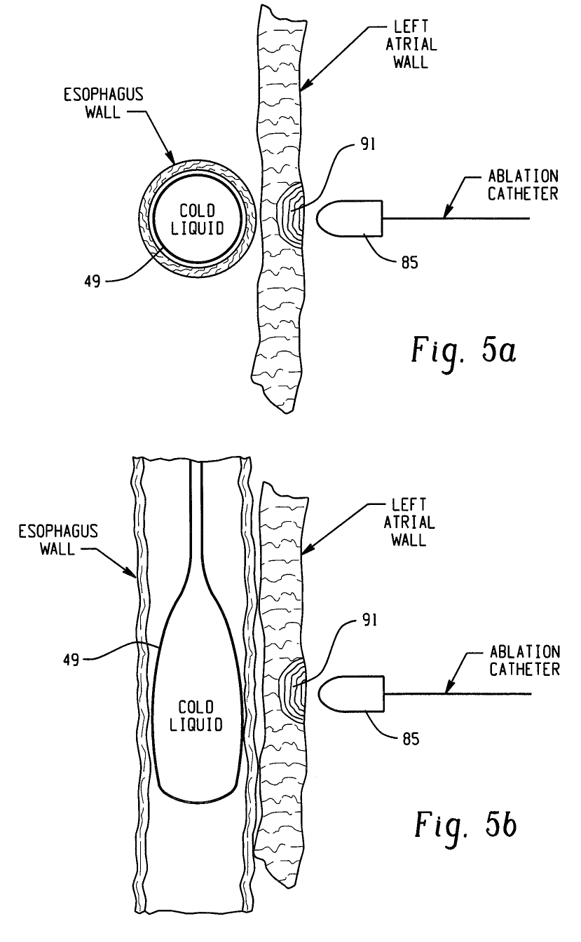

Referring now to FIG. 6, another embodiment of this invention illustrates an air probe that includes a flexible main tubular body or shaft 148 that comprises a perforated sac 108 with circular transversal cross-section at its distal portion. Two doughnut-shape inflatable sacs 114, 124 are disposed on the main body of the air probe 148 adjacent either side of said sac 108 with some spacing. The air probe further includes a perforated sac 108, ring-shape radio-opaque markers 142, 136, 138, 140, suction tube 100, cold air (gas) inlet tube 102 to the sac 108, air supplier tube 104 to the two doughnut-shape sacs 114, 124.

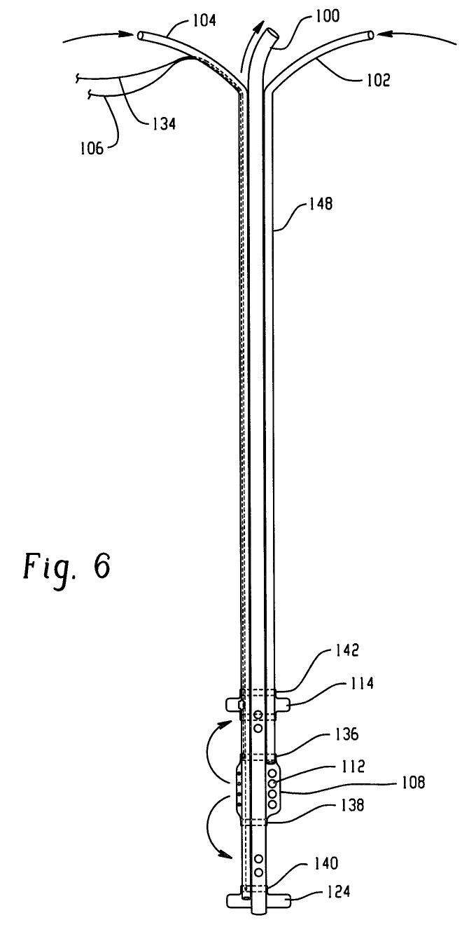

With continued reference to FIG. 6, and additional reference to FIG. 7, the air probe includes suction tube 100 with plurality of perforations (preferably at least four perforations 116, 118, 120 and 122), which are located between sac 108 and the inflatable doughnut-shape sacs 114 and 124. FIG. 7 further illustrates tube 102 and tube 104 within the main body of the air probe 148. As described with reference to FIG. 2b, cooling air/gas is supplied from tank 45 to the perforated sac 108 via tube 102. The two doughnut-shaped sacs 114 and 124 in FIG. 7 are inflated from the external air sources (i.e., syringe) via tube 104 and openings 115 and 126. Further, at least four platinum ring electrodes 142, 136, 138, and 140 serve as radio-opaque markers disposed on the sacs 108, 114, 124 to the main body 148 of the air probe. These ring electrodes can be used to identify the location of the sacs under fluoroscopy when the distal portion of the air probe is positioned in the esophagus. Preferably, at least one temperature sensor is disposed within the main body of the air probe 148 and attached to the ring electrodes.

FIG. 7 shows two temperature sensors 110 and 132. The air probe includes a flexible main body shaft 148, cooling air/gas supplier 102 to the sac 108, air/gas supplier (tube) 104 to the two doughnut-shape shaped inflatable sacs 114,124, radio-opaque markers 142, 136, 138, and 140, and two temperature sensors 110, 132. The suction tube 100 with multi-suction holes 116, 118, 120 and 122. Sac 108 consists of plurality of perforations (i.e., 112 and 128). During atrial ablation procedure the air probe 108 is positioned into the esophagus. The two doughnut-shaped sacs 114, 124 are inflated via tube 104 of the air probe to isolate a segment of the esophagus. Then, as illustrated in FIG. 2b, opening 56 of tank 45 is connected to a cold air (or gas) supply line. The pressure sensor 201 in tank 45 (FIG. 2b) monitors and assures that the desired regulated pressure is provided in the tank. The regulated cold air (gas) thereby flows to the perforated middle sac 108 of the air probe 148. This cold air (gas) flow reduces the temperature of the region of the esophagus where the middle sac 108 is positioned. Hot tank 43 could be used to elevate the temperature of the esophagus to the core body temperature if desired.

The suction tube 100 of the air probe 148 is connected to the flow meter 35. The suction pump 21 in FIG. 2b and FIG. 7 will evacuate trapped air (gas) between sac 108 and the two doughnut-shaped, inflated sacs 114 and 124 via the suction holes 116, 118, 120 and 122. The suction pump 21 of FIG. 2 then transfers the air (gas) to reservoir 47. The vent valve 9 on the reservoir is activated (opened), allowing continuous cold air to flow to the esophagus from the perforated sac 108 of probe 148.

A further embodiment of this invention provides a flexible magnetic probe that can be positioned into the esophagus via the nasal cavity. The flexible magnetic probe can be constructed by permanent magnet or a flow of electrical current to a magnetic coil within the main body of the probe. The magnetic segment of the flexible magnetic probe will have two different magnetic polarities on either ends. The flexible magnetic probe can be utilized during the atrial ablation procedure.

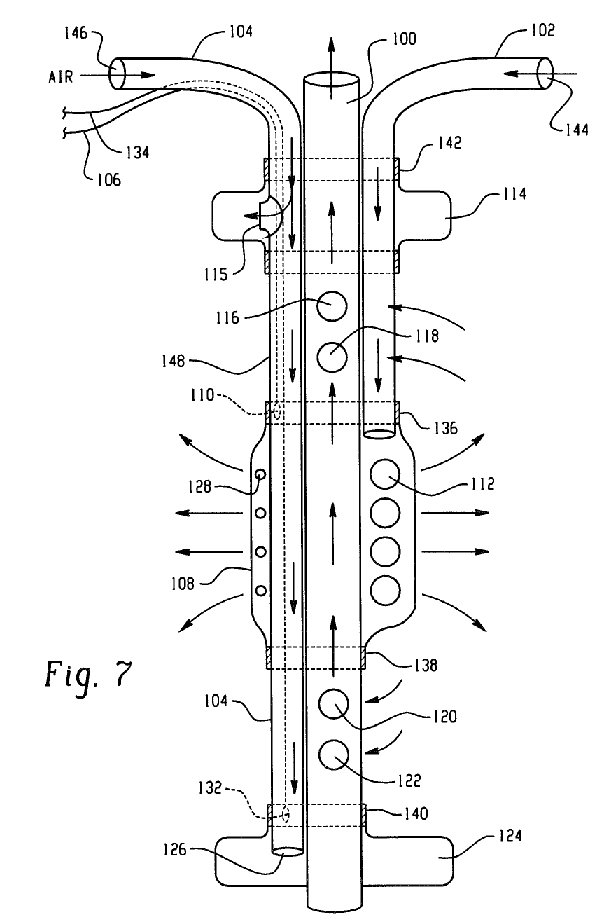

Referring to FIG. 8 of this invention, there is illustrated a preferred form of a flexible non-electrically conductive tubular probe 10. The NS segment of probe is either a flexible permanent magnet or an electrically activated magnet. The probe includes segment NS which is the magnet portion of the probe within tube 10. This segment can be constructed by a flexible permanent magnet or, again, a flow of electrical current via wires 6 and 8 extending from the magnetic coil within the main body 10 of segment NS of the probe. Two platinum ring electrodes 12 and 14 are disposed on a distal portion segment 24 of the probe and attached individually to wires 2 and 4 extending to the proximal end of the probe. These two electrodes can be used to pick up electrical activities of the heart from the esophagus side during an ablation procedure and help to identify the location of the left atrium. A temperature sensor 13 is interposed on the non-magnetic distal portion 24 of the probe preferably between the magnetic portion 22 and the ring electrodes. Again referring to FIG. 8 of this invention, segment 24 is the distal portion of the probe and is an extension of the main flexible tubular body 10 of the probe.

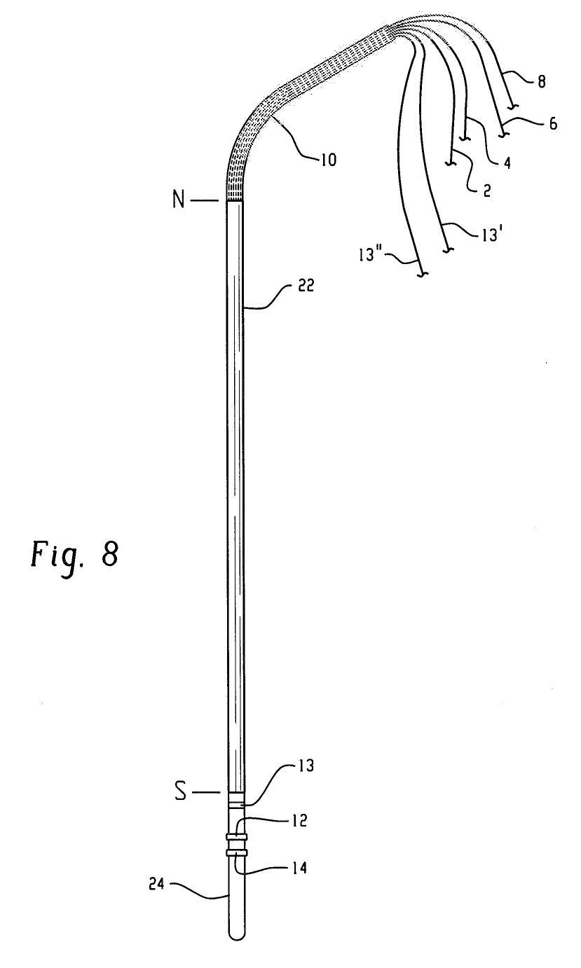

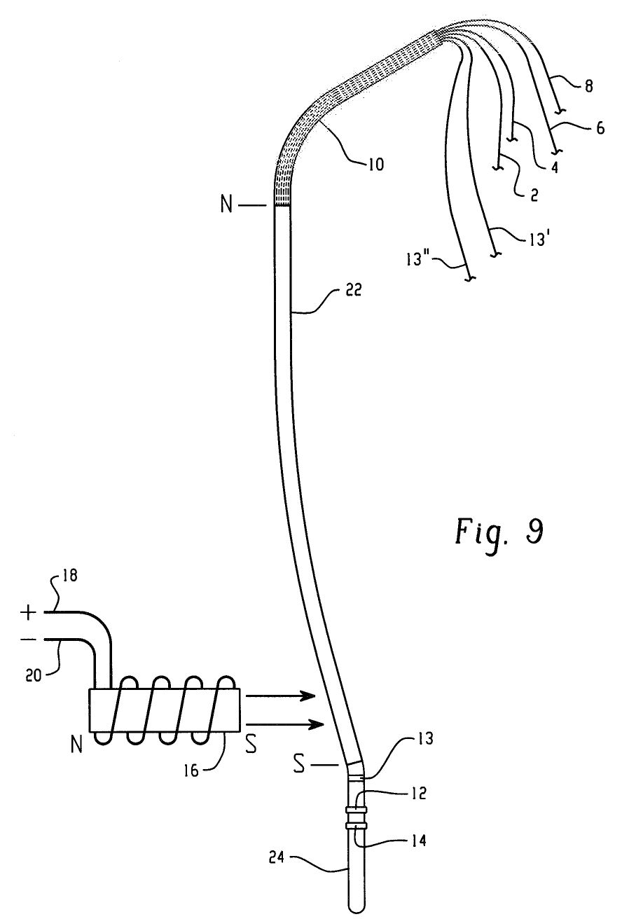

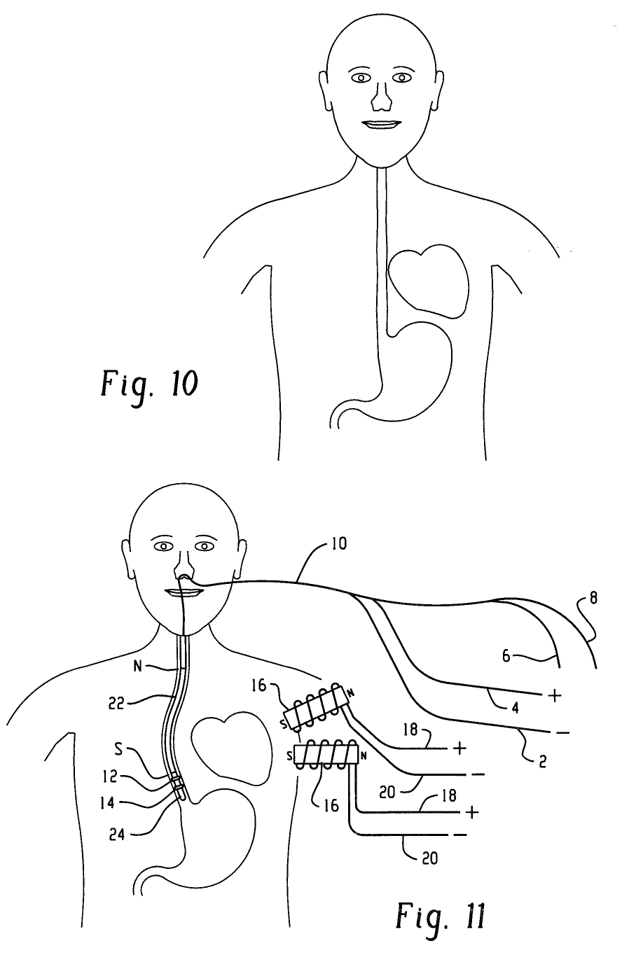

Referring to FIG. 9 of this invention, there is illustrated the exposure of a distal portion 24 of magnet segment of the probe 10 to a magnetic field of the same polarity that laterally deflects, displaces, or pushes the distal portion of the probe from the external magnetic source 16. Wires 13′ and 13″ extend through the length of the probe for connection with the temperature sensor 13. The impact of the ability to displace the distal portion of the probe laterally via a magnetic source is evident from a comparison of FIGS. 10 and 11. More specifically, FIG. 10 schematically shows a normal, relative location and position of the heart and esophagus. In FIG. 11, however, is shown a flexible magnetic probe 10 (such as described in FIGS. 8 and 9), that is placed into the esophagus via the nasal cavity. FIG. 11 also shows the distal magnetic end S of the probe. A segment of the probe 10 is positioned in the esophagus behind the left atrium. The distal end S of the probe is exposed to an external magnetic source of preferably a pair of sources 16 of the same polarity positioned on the left side of the patient chest. As FIG. 11 shows, the external magnetic field (or preferably fields when a pair of sources are used) pushes the distal end of the probe 10 resulting in temporary lateral displacement of the desired segment portion of the esophagus that would otherwise be in contact with the left atrium. The temporary separation of the esophagus from the left atrium can prevent a serious injury of the esophagus during the atrial ablation procedures. The pair of external magnetic fields provides greater control over the direction and displacement of the probe, for example, by disposing the magnetic fields in a converging fashion, i.e., at an angle relative to one another, so that a convergent magnetic force is applied to the probe in a desired direction.

Although the present invention has been described hereinabove with respect to the illustrated embodiments, it will be understood that the invention is capable of modification and variation and is limited only by the scope of the following claims.

1. A device comprising:
a probe having an elastic sac adjacent a distal end;
an inlet pump and first flow meter for directing fluid into the elastic sac; and
a suction pump and a second flow meter for suctioning fluid from the elastic sac to provide continuous circulation of fluid at a desired rate and desired sac inflation that could range from a non-inflated state to a fully inflated condition.

2. The device as defined in claim 1 wherein the circulating fluid flows through a desired non-inflated, semi-inflated, or fully inflated state of the elastic sac independent of the fluid pressure within the elastic sac.

3. The device of claim 1 wherein the sac is formed of an elastic material that as fluid increases in the sac, the volume increases to maintain a desired preselected pressure.

4. A device adapted for use with an associated ablation generator during a cardiac ablation procedure, the device comprising:
a probe having an elastic sac defined adjacent a distal end;
a temperature sensor that monitors a temperature in the sac;
an inlet pump and a first flow meter for directing liquid into the sac of the probe;
a suction pump and a second flow meter for suctioning liquid from the sac of the probe and continuously circulating liquid in the sac of the probe; and
a controller for receiving temperature data from the temperature sensor and selectively controlling power input to an associated ablation generator during the cardiac ablation procedure.

5. The device as defined in claim 4 wherein the probe is disposable and further includes a pressure sensor received in the sac.

6. The device as defined in claim 4 including a shutoff mechanism operatively associated with the controller for shutting off an associated ablation generator to prevent advancement of lesion formation during ablation energy delivery and protect a desired tissue depth in front of a created lesion by an associated tip of an associated ablation catheter.

7. The device as defined in claim 4 wherein the inlet pump provides a continuous flow of cooling fluid into the sac positioned at a desired region of an associated esophagus from an associated external cooling fluid source and the outlet pump returning fluid to the associated external source.

8. The device as defined in claim 4 wherein the probe includes a flexible magnetic portion dimensioned for receipt into an associated esophagus and adapted to temporarily displace a segment of the associated esophagus by exposing the magnetic portion of the probe to an external magnetic field.

9. The device as defined in claim 8 wherein the external magnetic field includes first and second converging magnetic fields.

10. A device for use with an associated ablation catheter system for protecting accidental esophagus injury in front of a lesion created by an associated ablation catheter tip, the device comprising:
a probe dimensioned for receipt in a patient's esophagus through a nasal cavity, the probe including an elastic sac adapted for locating the probe in the esophagus adjacent the left atrial wall;
an inlet pump and a first flow meter for providing a cooling medium to the sac and a suction pump and a second flow meter for suctioning the medium from the sac, the inlet and suction pumps providing a continuous flow of cooling fluid through the elastic sac; and
a temperature sensor operatively associated with the elastic sac and providing temperature data to the associated ablation catheter system.

11. The device of claim 10 further comprising a control unit receiving data from the temperature sensor indicative of the temperature of the esophagus wall when compared with input data from an ablation generator of the power supplied to the catheter tip.

12. The device of claim 10 further comprising a pressure sensor for monitoring pressure within the sac.

13. The device of claim 10 wherein a thermal gradient through the atrial wall is controlled by controlling the temperature in the esophagus at the location of the sac.

14. A method of preventing accidental injury of the esophagus during atrial ablation procedures in a patient's left atrium comprising:
placing a distal portion of a probe into a region of the esophagus in contact with the left atrium;
expanding an elastic sac of the probe to engage the esophagus region;
monitoring a desired temperature in the sac; and
controlling energy delivery from the ablation energy generator in response to the temperature in the sac to prevent advancement of lesion formation created by the ablation catheter in the left atrium.

15. The method of claim 14 further comprising supplying a continuous fluid through the sac and maintaining a desired temperature in the sac.

16. A device used in combination with an ablation generator during a left atrial ablation procedure comprising:
a probe having an elastic sac defined adjacent a distal end to be positioned into an esophagus;
an inlet pump and a first flow meter downstream thereof for providing fluid to the elastic sac;
a suction pump and a second flow meter upstream thereof for suctioning the liquid from the elastic sac;
a source of temperature regulating fluid that continuously circulates fluid through the sac in response to temperature data from a temperature sensor; and
a controller for receiving temperature data from the temperature sensor and selectively controlling power input to an ablation generator in response to the temperature data during the left atrial ablation procedure.

17. The device of claim 16 wherein the air probe includes radio-opaque markers.

18. The device of claim 16 wherein a distal portion of the probe includes a plurality of in-flow and out-flow perforations within tubes housed in the probe, the tubes extending to a proximal end of the probe that is connected to the related external source whereby cooling fluid with desired temperature and pressure is delivered from the external device to the out-flow perforations of the distal portion of the probe, and released fluid cools the desired region of the esophagus for return through the in-flow pores of the probe to the external source.

19. The device as defined in claim 16 wherein the inlet and suction pumps and associated first and second flow meters provide continuous circulation of liquid with desired rate and desired sac inflation that would range from a non-inflated state to a fully inflated state.

20. The device as defined in claim 18 wherein the circulating fluid flows through the elastic sac independent of the fluid pressure within the elastic sac.
